(19) United States
(12) Patent Application Publication
(10) Pub. No.: US 2024/0366823 A1
(43) Pub. Date: Nov. 7, 2024

(54) INSTANT ACTION AEROSOL FOR AIR SANITIZATION AND DISINFECTION

(71) Applicants: Reckitt Benckiser LLC, Parsippany, NJ (US); REXKITT BENCKISER (BRANDS ) lIMITED, Slough Berkshire (GB)

(72) Inventors: Martin Richard BUTLER, Causeway Bay (HK); Mohammad Khalid IJAZ, Montvale, NJ (US); Anthony KEVEK, Montvale, NJ (US)

(21) Appl. No.: 18/568,124

(22) PCT Filed: Jun. 17, 2022
(86) PCT No.: PCT/GB2022/051534

§ 371 Date: Dec. 7, 2023

Related U.S. Application Data
(60) Provisional application No. 63/217,102, filed on Jun. 30, 2021.

(30) Foreign Application Priority Data
Aug. 12, 2021 (GB) 2111603.3

(51) Int. Cl. A61L 9/01; A01P 1/00; A61L 9/14

(52) U.S. Cl. CPC A61L 9/01; A01P 1/00; A61L 9/14; A61L 2209/133; A61L 2209/134

(57) ABSTRACT

Air decontamination formulations are disclosed comprising approximately 28% to approximately 38% by weight of the formulation of dipropylene glycol; approximately 0.12% to approximately 0.43% by weight of the formulation of an odor neutralizing cationic surfactant; approximately 28% to approximately 50% by weight of the formulation of an alcohol, preferably ethanol; and approximately 14% to approximately 33% by weight of the formulation of water. The disclosed air decontamination formulations sanitize or disinfect a volume of air less than or equal to 25 m³ in 0.1 to 5 minutes by providing equal to or greater than a 3 log₁₀ reduction of aerosolized bacteria, including Staphylococcus aureus and Klebsiella pneumoniae, and aerosolized enveloped viruses, including Phi6.

### TECHNICAL FIELD

Air decontamination formulations are disclosed comprising approximately 28% to approximately 38% by weight of the formulation of dipropylene glycol; approximately 0.12% to approximately 0.37% by weight of the formulation of an odor neutralizing cationic surfactant; approximately 28% to approximately 50% by weight of the formulation of an alcohol, preferably ethanol; and approximately 14% to approximately 33% by weight of the formulation of water. The disclosed air decontamination formulations may sanitize or disinfect a volume of air less than or equal to 25 m³ in 0.1 to 5 minutes by providing equal to or greater than a 3 log₁₀ reduction of aerosolized bacteria, including Staphylococcus aureus and Klebsiella pneumoniae, and aerosolized enveloped viruses, including Phi6.

### BACKGROUND

Indoor air can be an important vehicle for spread of a variety of human pathogens, as evidenced by the most recent COVID-19 pandemic associated with severe acute respiratory coronavirus-2 (SARS-COV-2).

U.S. Pat. No. 2,333,124 to Robertson et al. discloses methods of sterilizing air by means of glycol vapors.

U.S. Pat. No. 2,719,129 to Richardson et al. discloses liquid room and air deodorant compositions comprising 0.5-5% of a quaternary morpholinium alkyl sulfate having an alkyl radical containing 8 to 24 carbon atoms as a deodorant, about 80-95% of a liquefied normally gaseous low molecular weight halogenated hydrocarbon propellent, and a sufficient amount of a partial ester of a polyhydric alcohol and a higher fatty acid having about 8 to 24 carbon atoms.

U.S. Pat. No. 8,465,728 to S.C. Johnson & Son, Inc. discloses an air treating composition for eliminating airborne malodors and/or sanitizing air in combination with a spray valve and actuator and spray performance parameters providing maximum dispersion of the composition. Col. 3, lines 21-23 states that no specific standards or methods for evaluating air sanitizers have been adopted by the US government.

Various analytical methods and air samplers have been used to characterize airborne pathogens and overcome the challenges of collecting and analyzing them. See, e.g., Satter et al., Spread of viral infections by aerosols, Crit Rev Environ Control, 1987, 17:89-131; Yates et al., Manual of environmental microbiology, 4^{th} Edition, Washington (DC): ASM Press: 2015 p. 3.2.7-1-24; Verreault et al., Design of an environmentally controlled rotating chamber for bioaerosol aging studies, Inhal Toxicol 2014; 26:554-8; Ijaz et al, Survival characteristics of airborne human coronavirus 229E, J Gen Virol., 1985 Dec, 66 (Pt 12), 2743-8; and Ijaz et al., Development of methods to study the survival of airborne viruses, J Virol Methods, 1987 Nov. 18 (2-3), 87-106.

In DIS/TSS-11 from 3 Sep. 1980, the US Environmental Protection Agency (EPA) advised that quantitative chemical determinations using an air sampling device must be performed on products containing at least 5% glycols (triethylene, dipropylene, and/or propylene glycols) to show that 50% glycol vapor saturation or more is achieved in the enclosed experimental room or chamber.

The EPA's Office of Chemical Safety and Pollution Prevention (OCSPP) issued guidance to address efficacy testing for antimicrobial pesticides intended to be used for treatment of air to temporarily reduce the number of airborne bacteria on 21 Dec. 2012 as OCSU.S. Plant Pat. No. 810.2500. The guidance states that successful test results demonstrate a viable bacteria count reduction of ≥99.9 percent (a 3 log₁₀ reduction) over a parallel untreated control, after correcting for settling rates, in the air of the test enclosure with each of the required test bacteria.

Both EPA guidelines also note that there is no standard test method for evaluating effectiveness of antimicrobial pesticides to temporarily reduce airborne bacteria.

A need remains to develop microbicidal formulations demonstrating efficacy against airborne pathogens.

### BRIEF SUMMARY

Air decontamination formulations are disclosed comprising or consisting essentially of approximately 28% to approximately 38% by weight of the formulation of dipropylene glycol; approximately 0.12% to approximately 0.43% by weight of the formulation of an odor neutralizing cationic surfactant; approximately 28% to approximately 50% by weight of the formulation of an alcohol; and approximately 14% to approximately 33% by weight of the formulation of water. The disclosed air decontamination formulations may comprise one or more of the following aspects:
the formulation being a single phase liquid;
the formulation containing a ratio of alcohol: water of approximately 1.5:1;
the formulation containing a ratio of dipropylene glycol: water: alcohol of approximately 1.25:1:1.5;
the formulation containing a ratio of alcohol: water of approximately 1.5:1 and packaged for aerosol dispensing using a hydrocarbon propellant;
the formulation containing a ratio of dipropylene glycol: water: alcohol of approximately 1.25:1:1.5 and being packaged for aerosol dispensing using a hydrocarbon propellant;
the propellant being a blend of propane and isobutane;
the propellant providing at least 60 psig pressure;
the odor neutralizing cationic surfactant being a quaternary morpholinium alkyl sulfate compound;
the quaternary morpholinium alkyl sulfate compound being selected from the group consisting of soyethyl morpholinium ethosulfate, cetethyl morpholinium ethosulfate, N-myristyl-N-methyl morpholinium methyl sulfate, and N-oleyl-N-methyl morpholinium methyl sulfate;
the quaternary morpholinium alkyl sulfate compound being soyethyl morpholinium ethosulfate and/or cetethyl morpholinium ethosulfate;
the quaternary morpholinium alkyl sulfate compound being N-myristyl-N-methyl morpholinium methyl sulfate and/or N-oleyl-N-methyl morpholinium methyl sulfate;
the quaternary morpholinium alkyl sulfate compound being soyethyl morpholinium ethosulfate;
the quaternary morpholinium alkyl sulfate compound being cetethyl morpholinium ethosulfate;
the quaternary morpholinium alkyl sulfate compound being N-myristyl-N-methyl morpholinium methyl sulfate;
the quaternary morpholinium alkyl sulfate compound being N-oleyl-N-methyl morpholinium methyl sulfate;
the air decontamination formulation further comprising a corrosion inhibitor; the air decontamination formulation further comprising approximately 0.043% to approximately 0.43% by weight of the formulation of a corrosion inhibitor;
the air decontamination formulation further comprising approximately 0.043% to approximately 0.43% by weight of the formulation of a pH adjuster;
the air decontamination formulation optionally further comprising 0% to approximately 0.28% by weight of the formulation of a defoaming agent;
the air decontamination formulation further comprising approximately 0.05% to approximately 0.43% by weight of the formulation of a dye, fragrance, or both;
the air decontamination formulation not including an emulsifier;
the air decontamination formulation not including a nonionic surfactant; and/or.
the air decontamination formulation not including an amphoteric surfactant.

Air decontamination formulations are also disclosed consisting of approximately 28% to approximately 38% by weight of the formulation of dipropylene glycol; approximately 0.12% to approximately 0.43% by weight of the formulation of an odor neutralizing cationic surfactant; approximately 28% to approximately 50% by weight of the formulation of an alcohol, preferably ethanol; approximately 14% to approximately 33% by weight of the formulation of water; approximately 0.043% to approximately 0.43% by weight of the formulation of a corrosion inhibitor; approximately 0.043% to approximately 0.43% by weight of the formulation of a pH adjuster; optionally 0% to approximately 0.28% by weight of the formulation of a defoaming agent; and optionally 0% to approximately 0.43% by weight of the formulation of dye, fragrance, or both.

Methods of sanitizing and disinfecting air in 0.1 to 5 minutes are also disclosed. Between approximately 0.825 g/second to approximately 1.125 g/second of any of the air decontamination formulations disclosed above is introduced into the air for approximately 30 to approximately 60 seconds, the air occupying a volume being less than or equal to 25 m³. The air decontamination formulation may be introduced into the air in one continuous 30 to 60 second spray. Alternatively, the air decontamination formulation may be introduced into the air in multiple consecutive sprays that combine to total 30 to 60 seconds of spray in a total time of less than 35 to 70 seconds.

Air decontamination products are also disclosed. The air decontamination products comprise an aerosol canister containing a 2-piece mechanical breakup (MB) nozzle with swirl chamber, 70-80% by weight of the combined decontamination formulation and pressurized liquefied petroleum gas (LPG) of any of the decontamination formulations disclosed above, and 20-30% by weight of combined decontamination formulation and LPG of the pressurized liquified petroleum gas, the pressurized liquified petroleum gas comprising a blend of propane and butane sufficient to produce at least 60 psig pressure; and the MB nozzle comprising a housing orifice having a diameter ranging from approximately 0.02 inches [0.5 mm] to approximately 0.03 inches [0.8 mm], a stem vapor tap having a diameter ranging from approximately 0.016 inches [0.41 mm] to approximately 0.020 inches [0.51 mm], and a quantity of two [2] or four [4] stem orifices 110 having a diameter ranging from approximately 0.024 inches [0.61 mm] to approximately 0.025 inches [0.64 mm]. The LPG may comprise approximately 42.89% weight of the gas of propane and approximately 57.11% weight of the gas of isobutane.

Another method of sanitizing and disinfecting air in 0.1 to 5 minutes is disclosed. The MB nozzle of the air decontamination products disclosed above is actuated to introduce between approximately 1.1 g/second to approximately 1.5 g/second of the air decontamination formulations disclosed above into the air for approximately 30 to approximately 60 seconds, the air occupying a volume being less than or equal to 25 m³. The air decontamination formulation may be introduced into the air in one continuous 30 to 60 second spray. Alternatively, the air decontamination formulation may be introduced into the air in multiple consecutive sprays that combine to total 30 to 60 seconds of spray in a total time of less than 35 to 70 seconds.

The above embodiments are exemplary only. Other embodiments as described herein are within the scope of the disclosed subject matter.

### BRIEF DESCRIPTION OF THE SEVERAL VIEWS OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a diagram of the 2-piece MB nozzle aerosol valve assembly that may be used in the present invention;

FIG. 2 is a diagram of liquid electrical vaporizer that may be used in the present invention;

FIG. 3 is a perspective view of an aerobiological testing chamber used in the examples that follow;

FIG. 4 is a flow chart of the steps performed in the following examples;

FIG. 5 is a graph of the log₁₀ concentration of the tested microorganism versus time; and

FIG. 6 is a graph of the log₁₀ concentration of the tested microorganism versus time.

### DEFINITIONS

As used herein, the term “a” or “an” means one or more.

As used herein, the term “comprising” is inclusive or open-ended and does not exclude any additional elements; the term “consisting of” excludes any additional elements; and the term “consisting essentially of” is in-between, only permitting additional elements that do not materially affect characteristics of the product or process.

As used herein, the term “approximately” or “about” means plus or minus 10 percent of the value stated.

As used herein, the w/w percent of an ingredient is based on the weight of the ingredient in grams in the total weight of the formulation in grams. When an ingredient does not contain close or equal to 100% active material, two percentages may be provided: one for the weight of the ingredient and one for the weight of the active. For example, 0.2 g of Forestall-LQ-(HM) sold by Croda contains approximately 35% soyethyl morpholinium ethosulfate in water, alcohol and/or polypropylene glycol, which equates to approximately 0.07 g of soyethyl morpholinium ethosulfate in the formulation. Both concentrations are represented as 0.2 g [0.07 g] of odor neutralizing cationic surfactant.

As used herein, any and all ranges are inclusive of their endpoints. For example, a pH ranging from 10.5 to 11.2 would include formulations having a pH of 10.5, formulations having a pH of 11.2, and formulations having any pH between 10.5 and 11.2.

As used herein, the terms “germ” and “microbe” or “microbial” means microorganisms which causes disease and encompasses both bacteria and viruses and “microbicidal” means formulations that inactivate (or kill) germs and microbes.

As used herein, the terms “decontamination” or “decontaminate” mean to reduce the concentration of aerosolized microbes in the air. The terms “sanitize”, “disinfect,” “sanitization,” and “disinfection” mean providing equal to or greater than a 3 log₁₀ reduction in 0.1 to 5 minutes of aerosolized microbes, including Staphylococcus aureusand Klebsiella pneumoniae, and aerosolized enveloped viruses, including Phi6, in air occupying a volume less than or equal to 25 m³.

As used herein, the term “swirl chamber” may also be known as a spin chamber.

### DETAILED DESCRIPTION

Glycol vapors have been shown to produce decreases in numbers of viable airborne bacteria within enclosed spaces. See, e.g., the US EPA's Efficacy Data and Labeling Requirements: Air Sanitizers, DIS/TSS-11, 3 Sep. 1980; Robertson et al., A Study of the Bactericidal Activity in vitro of Certain Glycols and Closely Related Compounds, The Journal of Infectious Diseases, Vol. 83, No. 2 (September-October 1948), pp. 124-137. The product label for SC Johnson's Oust™ surface disinfectant and air sanitizer lists 6.0% triethylene glycol as one of its active ingredients. As demonstrated in the examples that follow, this triethylene glycol concentration is unlikely to provide air sanitization as defined herein.

Triethylene glycol (TEG) is a colorless, odorless viscous liquid. TEG has a boiling point of 285° C. and density of 1.1255 g/mL. TEG has a vapor pressure of 0.02 Pa at 20° C. TEG is soluble in ethanol and miscible with water. PCT Pub No. WO2007/117534 to SC Johnson (the '534 Publication) discloses that ethanol must be added to TEG-based aerosol compositions to increase its solubility in hydrocarbon propellants. The '534 Publication further discloses that quantities of 12-15% triethylene glycol would produce a two-phase system requiring vigorous shaking by consumers before use.

Applicants have surprisingly discovered that air decontamination formulations comprising or consisting essentially of approximately 28% w/w to approximately 43% w/w dipropylene glycol, approximately 0.12% w/w to approximately 0.43% w/w odor neutralizing cationic surfactant, approximately 28% w/w to approximately 50% w/w alcohol, and approximately 14% w/w to approximately 33% water provide air disinfection.

These formulations are a single liquid phase and therefore do not require shaking by consumers before use. Single phase formulations are also typically easier to manufacture, vaporize and exhibit less stability issues than two phase formulations. Specifically, the ingredients that produce the disclosed air decontamination formulations may be mixed in one pot, reducing manufacturing complexity. The resulting formulations have a water-like viscosity (i.e., 0.89 mPa·s at 25°° C.).

Dipropylene glycol (DPG) is a colorless, nearly odorless liquid. DPG has a boiling point of 227° C. and a viscosity of 1.02 g/mL at 20° C. DPG has a vapor pressure of 2.7 Pa at 20° C. DPG's vapor pressure increases to about 250 Pa just below 100° C. DPG is soluble in ethanol and water. DPG is a mixture of three isomeric chemical compounds: 4-oxa-2,6-heptandiol, 2-(2-hydroxy-propoxy)-propan-1-ol, and 2-(2-hydroxy-1-methyl-ethoxy)-propan-ol. One of ordinary skill in the art will recognize that different isomers may have different properties. Applicants have found that DPG produced by non-catalytic hydration reaction with propylene oxide and water followed by vacuum distillation produces a consistent isomer profile.

The air decontamination formulations comprise from approximately 28% by weight to approximately 43% by weight dipropylene glycol, preferably from approximately 28% by weight to approximately 38% by weight, more preferably from approximately 31% by weight to approximately 38% by weight, more preferably from approximately 33% by weight to approximately 37% by weight, and most preferably from approximately 34% by weight to approximately 36% by weight.

The U.S. Environmental Protection Agency's Office of Pollution Prevention and Toxics expected volatilization of DPG from water and aqueous solutions to be minimal based on DPG's estimated Henry's Law constant (2020 Feb. 20: Supporting Information for Low-Priority Substance Propanol, Oxybis (CASRN 25265-71-8) (Dipropylene Glycol) Final Designation at Section 3 on page 7). Surprisingly contrary to the EPA's characterization, the claimed combination of DPG, odor neutralizing cationic surfactant, alcohol, and water provides sufficient air saturation to reduce airborne germ load and even sanitize the air as shown in the examples that follow.

The air decontamination formulations comprise from approximately 0.07% by weight to approximately 0.29% by weight of an odor neutralizing cationic surfactant, preferably from approximately 0.11% by weight to approximately 0.21% by weight. Exemplary odor neutralizing cationic surfactants include, but are not limited to, quaternary morpholinium alkyl sulfate compounds, such as soyethyl morpholinium ethosulfate, cetethyl morpholinium ethosulfate, N-myristyl-N-methyl morpholinium methyl sulfate, N-oleyl-N-methyl morpholinium methyl sulfate, or combinations thereof. Soyethyl morpholinium ethosulfate and/or cetyl ethyl morpholinium ethosulfate are particularly preferred odor neutralizing cationic surfactants. Exemplary commercial sources of soyethyl morpholinium ethosulfate include the product sold under the tradename Forestall-LQ-(HM) sold by Croda. Exemplary commercial sources of cetyl ethyl morpholinium ethosulfate include the product sold under the tradename Barquat cme-A by Lonza.

As stated above, DPG is soluble in both water and ethanol. The percentage of water and alcohol in the disclosed air decontamination formulations is important to obtain optimal vaporization of both the DPG and odor neutralizing cationic surfactant. Applicants believe that air disinfection and odor removal will be obtained from air decontamination formulations containing (a) a combination of between approximately 28% w/w to approximately 50% w/w alcohol and approximately 33% to approximately 14% water and (b) a suitable dispensing device. In aerosol dispensing applications using a hydrocarbon gas, a combination of between approximately 36% w/w to approximately 45% w/w alcohol and approximately 29% to approximately 21% water is particularly preferred, more preferably between approximately 36% w/w to approximately 43% w/w alcohol and approximately 27% w/w to approximately 25% w/w water. Preferably, the formulation contains a weight ratio of alcohol: water of approximately 1.5:1 for aerosol applications. Preferably, the formulation contains a weight ratio of DPG: water: alcohol of approximately 1.25:1:1.5 for aerosol applications. One of ordinary skill in the art will recognize that the propellant helps to both dry the droplets being dispensed and aerosolize the formulation. Non-aerosol applications will need to be formulated to compensate for DPG's low vapor pressure and to remove the drying effect provided by the aerosol. As a result, a combination of between approximately 7% w/w to approximately 12% w/w alcohol and approximately 0.5% to approximately 4% water may be provided for any dispensing devices that do not utilize a propellant. For non-aerosol applications, the formulation preferably contains a ratio of alcohol: water of approximately 10:1 to approximately 3:1.

Due to its safety profile, ethanol is the preferred alcohol. However, a combination of C1-C6 alcohols may be used in the disclosed air decontamination formulations without departing from the teachings herein. For example, the quantity of alcohol in the formulation may comprise 95% ethanol and 5% tert-butyl alcohol or 98% ethanol and 2% isopropanol (e.g., approximately 14% by weight of the formulation to approximately 47% by weight of the formulation ethanol and approximately 1% by weigh of the formulation to approximately 3% by weight of the formulation t-butyl alcohol). One of ordinary skill in the art will recognize that both alcohols and propellants are volatile organic compounds (VOCs) and therefore the concentrations of both are also selected to remain within any governmental VOC limits.

The disclosed air decontamination formulations do not require any emulsifiers or amphoteric or nonionic surfactants because the ingredients combine to form a single liquid phase. For example, the disclosed air decontamination formulations do not contain any partial esters of a polyhydric acid, alcohol or ether, such as glycerine monostearate, sorbitol monostearate, propylene glycol monostearate, or diethylene glycol monostearate. The discloses air decontamination formulations also do not include glyceryl dilaurate, ethylene glycol monopalmitate, propylene glycol monolaurate, ethylene glycol monostearate, or propylene glycol monopalmitate. One of ordinary skill in the art will recognize that fragrances may contain trace amounts of these ingredients. However, any trace amounts included in any fragrances are utilized for the fragrance itself and would not provide sufficient quantities to affect the properties of the disclosed air decontamination formulations. As a result, the disclosed formulations may contain between 0% and approximately 0.1% by weight of a combination of any emulsifiers and amphoteric and nonionic surfactants, more preferably between 0% and approximately 0.075% by weight, and most preferably between 0% and approximately 0.05% by weight.

The disclosed air decontamination formulations further comprise a pH adjuster to maintain a pH ranging from approximately 10.5 to approximately 11.2. Exemplary pH adjusters include but are not limited to inorganic bases, alkanolamines, or combinations thereof. Exemplary inorganic bases include hydroxides, such as sodium hydroxide, potassium hydroxide, magnesium hydroxide, ammonium hydroxide, or combination thereof. Exemplary alkanolamines include monoethanolamine, diethanolamine triethanolamine, methylethylhydroxypropylhydroxylamine, or combinations thereof. Buffers may also be used as the pH adjuster, such as glycine and sodium hydroxide, ammonia and ammonium chloride, or sodium dihydrogen phosphate and sodium hydroxide.

When provided in a metal or alloy container, the disclosed air decontamination formulations may optionally further comprise a corrosion inhibitor. The corrosion inhibitor decreases the reaction between the air decontamination formulation and the metal or alloy container. The same ingredient may provide both the buffering and corrosion inhibitor capabilities to the formulation. The disclosed air decontamination formulations may optionally comprise between approximately 0.03% by weight to approximately 0.3% by weight corrosion inhibitor. Exemplary corrosion inhibitors include but are not limited to sodium hydroxide, sodium benzoate, sodium nitrite, sodium silicate, sodium lauryl sarcosinate, borates, or combinations thereof. Exemplary borate corrosion inhibitors may comprise a mixture of monoethanolamine (MEA) borate and monoisopropanol amine (MIPA) borate. An exemplary suitable commercial source of the MEA/MIPA borate corrosion inhibitor is sold under the tradename Crodacor™ BE by Croda.

Alternatively, the metal or alloy container may include an interior coating that prevents reaction between the metal or alloy container and its contents. Exemplary coatings include but are not limited to silicon oxide coatings.

Aerosol formulations frequently include a defoaming agent to prevent foaming. Defoaming agents may also be used to solubilize other ingredients, such as the fragrance. The disclosed formulations do not produce a lot of foam and therefore the defoaming agent is not mandatory, unless required for a specific fragrance. The disclosed air decontamination formulations may optionally comprise between 0% to approximately 0.28% by weight defoaming agent. Exemplary defoaming agents include but are not limited to PEG-12 Dimethicone. An exemplary suitable commercial source of PEG-12 dimethicone is sold under the tradename Xiameter™ OFX-0193 Fluid by Dow.

The disclosed air decontamination formulations may optionally further include dye, fragrance, or both.

A particularly preferred air decontamination formulation consists of approximately 28% by weight to approximately 43% by weight of the formulation of dipropylene glycol; approximately 0.12% by weight to approximately 0.43% by weight of the formulation of an odor neutralizing cationic surfactant; approximately 28% by weigh to approximately 50% by weight of the formulation of an alcohol, preferably ethanol; approximately 14% by weigh to approximately 33% by weight of the formulation of water; optionally 0% by weight to approximately 0.43% by weight of the formulation of a corrosion inhibitor; optionally 0% by weight to approximately 0.43% by weight of the formulation of a pH adjuster; optionally 0% by weight to approximately 0.29% by weight of the formulation of a defoaming agent; and optionally 0% by weight to approximately 0.43% by weight of the formulation of dye, fragrance, or both.

The disclosed air decontamination formulations may be packaged in a single use or multi-use aerosol container. Alternatively, the disclosed air decontamination formulation may be packaged in non-aerosol containers and vaporized using mechanical devices, such as piezoelectric vaporizers. As discussed above, adjustment of the alcohol and water concentrations in the disclosed air decontamination formulations will be necessary for non-aerosol applications due to the low vapor pressure of DPG and drying effect provided by the aerosol.

The disclosed air decontamination formulations reduce the concentration of germs in the air and may sanitize and disinfect air. For testing purposes, the air may be contained in an area less than or equal to 25 m³. One of ordinary skill in the art will recognize that air volume is only provided for comparative testing purposes and that the disclosed formulations may be used in larger areas without departing from the teachings herein. Between approximately 1.1 g/second to approximately 1.5 g/second of the aerosolized air decontamination formulations disclosed above is introduced into the air for approximately 30 to approximately 60 seconds. The air decontamination formulations may be introduced into the air in one (1) 30-60 second spray or multiple sequential shorter sprays.

As shown in the Examples that follow, the disclosed air decontamination formulations provide a 3 log₁₀ reduction of Staphylococcus aureus, Klebsiella pneumoniae, and Phi6. S. aureus is one of the more difficult bacteria to eradicate. K. pneumoniae is one of the easier bacteria to eradicate. Phi6 is a bacteriophage that acts as a testing surrogate for enveloped viruses, like SARS-COV-2, corona and influenza viruses. The disclosed air decontamination formulations may also be used to reduce the concentration of small non-enveloped viruses, as demonstrated in the examples that follow with the MS2 bacteriophage. One of ordinary skill in the art will recognize that non-enveloped viruses are more difficult to inactivate than bacteria and enveloped viruses. As a result, the disclosed air decontamination formulations take longer to inactivate MS2 than S. aureus, K. pneumoniae, and Phi6.

As discussed above, the air decontamination formulation may be packaged in an aerosol canister. The aerosol container is loaded with the liquid air decontamination formulation and propellant to a pressure approximately equal to or slightly greater than the vapor pressure of the propellant. The resulting air decontamination product comprises an aerosol canister containing a 2-piece mechanical breakup (MB) nozzle with swirl chamber, approximately 70% by weight to approximately 80% by weight of the combined weight of the pressurized liquefied petroleum gas (LPG) and air decontamination formulation of the air decontamination formulations disclosed above, and approximately 20% by weight to approximately 30% by weight of the combined weight of the LPG and air decontamination formulation of the LPG. The pressurized liquified petroleum gas comprises a blend of propane and butane sufficient to produce at least 60 psig pressure, preferably from about 70 psig to about 80 psig. For example, the pressurized liquified petroleum gas may comprise approximately 40% to approximately 60% by weight of the propane gas and approximately 40% to approximately 60% by weight of the isobutane gas. One exemplary gas suitable for use with the teachings herein has 42.89% weight propane and 57.11% weight isobutane. This gas is commercially available from multiple vendors as the A-70 hydrocarbon blend having a 70 psig pressure.

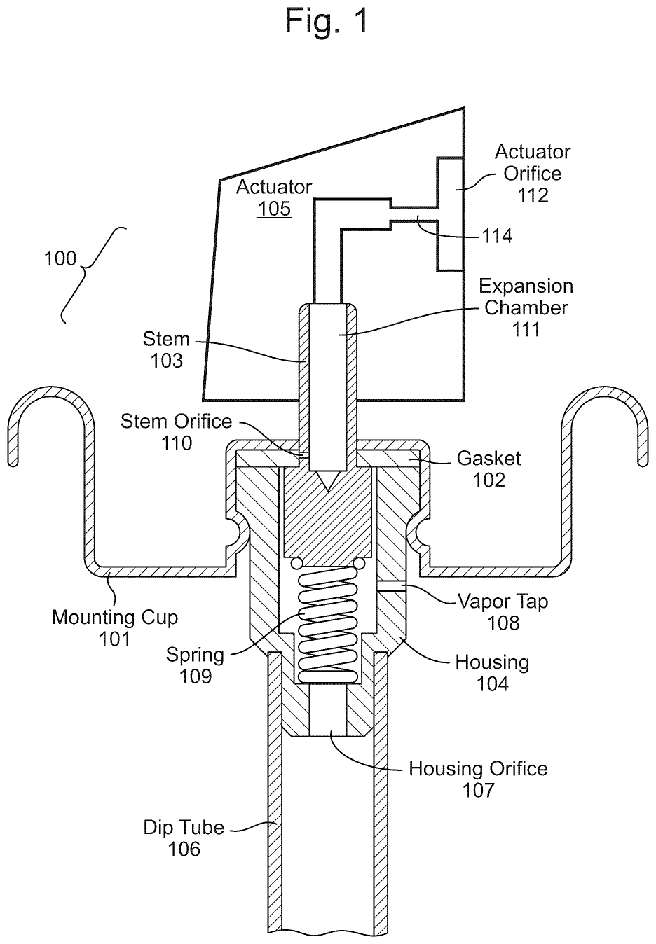

FIG. 1 is a diagram of an exemplary 2-piece MB nozzle aerosol valve assembly 100 that may be used to dispense the disclosed air decontamination formulations. The valve assembly 100 is connected to the mounting cup 101. The mounting cup 101 is crimped to the aerosol canister (not shown) in an air-tight manner. A gasket 102 seals the junction between the top of the valve assembly housing 104, mounting cup 101, and stem 103. The stem 103 extends from the valve assembly housing 104 through the gasket 102 and mounting cup 101 to the actuator 105. The valve assembly housing 104 includes a housing orifice 107, vapor tap 108, and spring 109. The spring 109 holds the valve assembly housing 104 in the closed position. The stem 103 includes a stem orifice 110 and an expansion chamber 111. The actuator 105 includes a swirl chamber 114 in fluid connection with an actuator orifice 112. The actuator 105 in FIG. 1 depicts a right angle from the stem 103 to the actuator orifice 112. One of ordinary skill in the art will recognize that a straight pathway may also be used without departing from the teachings herein. A dip tube 106 is connected to the bottom of the valve assembly housing 104.

When the actuator 105 is depressed, the stem 103 moves downward, which opens the seal between the gasket 102 and the stem orifice 110. The propellent forces the disclosed air decontamination formulations into the dip tube 106 and through the housing orifice 107 into the valve assembly housing 104. Propellant introduced into the valve assembly housing 104 through the vapor tap 108 mixes with the disclosed air decontamination formulation inside the valve assembly housing 104. The propellant both dries the air decontamination formulation as well as begins formation of aerosol droplets and subsequent glycol vapors. The generation of glycol vapors is critical in the denaturation of the airborne microbiological species. Applicants have found that the propellant must have at least 60 psig pressure in order to vaporize the disclosed air decontamination formulations. The air decontamination formulation/propellant blend moves from the valve assembly housing 104 through the stem orifice 110 into the expansion chamber 111. From the expansion chamber 111, the formulation moves through the actuator 105 to the swirl chamber 114 and out the actuator orifice 112 as an aerosolized spray.

Numerous swirl chambers 114 are commercially available. See, e.g., U.S. Pat. No. 3,583,642 to SC Johnson & Son, Inc., the contents of which are incorporated herein in its entirety by reference. The swirl chamber 114 is one factor in the production of aerosol particles of the desired size. Initial R&D tests conducted without a swirl chamber 114 resulted in visible spray on the floor below the nozzle. One of ordinary skill in the art will recognize that a pin orifice may also produce a suitable vapor.

The size of the vapor tap 108 is another factor that helps determine the size of the aerosol particles. Decreasing the size of the vapor tap 108 lowers the ratio of the propellant to air decontamination formulation and reduces the amount of formulation retention in the canister. But decreasing the size of the vapor tap 108 also increases the aerosol particle size, which may prevent aerosolization of the formulation due to the low vapor pressure of DPG. In other words, as shown in the examples that follow, too large an aerosol particle size of the disclosed air decontamination formulations results in liquid being visible on the floor below the canister nozzle. Liquid on the floor does not provide effective air sanitization.

The size of the housing orifice 107 also contributes to the size of the aerosol particles. Decreasing the size of the housing orifice 107 decrease the aerosol particle size.

The size of the stem orifice 110 also contributes to the size of the aerosol particles. Decreasing the size of the stem orifice 110 decrease the aerosol particle size.

The 2-piece MB nozzle aerosol valve assembly 100 comprises a housing orifice 107 having a diameter ranging from approximately 0.02 inches [0.5 mm] to approximately 0.03 inches [0.8 mm], a vapor tap 108 having a diameter ranging from approximately 0.016 inches [0.41 mm] to approximately 0.020 inches [0.51 mm], and a quantity of two [2] or four [4] stem orifices 110 having a diameter ranging from approximately 0.024 inches [0.61 mm] to approximately 0.025 inches [0.64 mm].

The aerosol particles produced by the aerosol valve assembly 100 have a particle size ranging from between approximately 1 micron and approximately 40 microns, preferably with less than 10% being less than 10 microns in diameter.

As shown in the Examples that follow, the combination of the disclosed air decontamination formulations and the 2-piece MB nozzle aerosol valve assembly 100 sanitizes air in 0.1 to 5 minutes. The actuator 105 is depressed for approximately 30 seconds to approximately 60 seconds to introduce between approximately 1.1 g/second to approximately 1.5 g/second of the air decontamination formulation into a volume of air occupying less than or equal to 25 m³. The actuator 105 may be depressed manually. Alternatively, the actuator 105 may automatically remain depressed for the desired dispensing duration. For example, the valve assembly 100 may include a solenoid switch as disclosed in PCT Publication Nos. WO2007/045826, WO2007/045827, and WO2007/045828, the contents of which are incorporated herein by reference in their entireties. More particularly, the valve assembly 100 may comprise a moveable magnetic stem 103 surrounded by copper windings (not shown), with an iron frame (not shown) surrounding the copper windings. Electric current applied to the copper windings moves the magnetic stem 103 to either the open or closed position.

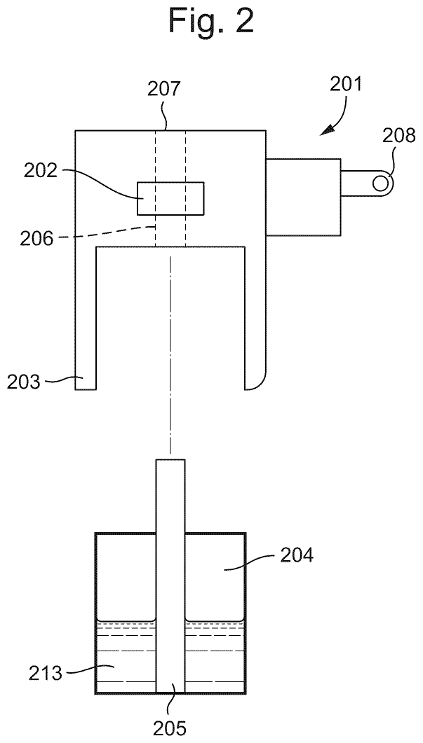

Alternatively, vaporization of the disclosed non-aerosol air decontamination formulations may occur using increased temperature delivered by a liquid electrical vaporizer. The non-aerosol air decontamination formulations comprise or consist essentially of between approximately 84% to approximately 92.5% by weight dipropylene glycol, between approximately 7% to approximately 12% w/w alcohol and approximately 0.5% to approximately 4% by weight water. An exemplary liquid electric vaporizer 201 is shown in FIG. 2, borrowed from FIG. 2 of PCT Pub. No. WO2019/038529 to Reckitt Benckiser (Brands) Limited, the contents of which are incorporated herein in its entirety by reference. The liquid electrical vaporizer 201 typically consists of a housing 203 with electrical plug 208 and a heater 202 and a detachable refill bottle 204 with a wick 205 or other liquid transfer mechanism, such as a ceramic coil. The wick 205 may form part of the refill bottle 204. The disclosed air decontamination formulations 213 are contained in the detachable refill bottle 204. The detachable refill bottle 204 typically holds between approximately 20 mL and approximately 25 mL of air decontamination formulation 213, but may easily be adjusted by one of ordinary skill in the art to hold between approximately 100 mL to approximately 500 mL to achieve the purposes disclosed herein. One of ordinary skill in the art will recognize that the single phase nature of the disclosed air decontamination formulations, and the stability associated therewith, make them particularly suitable for liquid electrical vaporization. Upon insertion of the refill bottle 204 into the housing 203, the wick 205 or ceramic coil is situated close to the heater 202. When the liquid electrical vaporizer 201 is plugged into an electrical outlet, the heater 202 is activated to provide emanation of the air decontamination formulation. The liquid electrical vaporizer 201 of FIG. 2 further comprises a chimney 206. The chimney 206 has an opening 207 through which the vaporized active leaves the vaporizer 201. As discussed above, air sanitization and disinfection occurs when between approximately 1.1 g/second to approximately 1.5 g/second of the aerosolized air decontamination formulation is introduced into the air for approximately 30 to approximately 60 seconds. Without the aerosol, air sanitization and disinfection is expected to occur when approximately 0.825 g/sec to approximately 1.125 g/sec of the non-aerosolized decontamination formulation is introduced in to the air for approximately 30 to approximately 60 seconds. Non-aerosol applications will need to be formulated to compensate for DPG's low vapor pressure and to remove the requirement for drying provided by the aerosol. As a result, may be provided for any dispensing devices that do not utilize a propellant. For non-aerosol applications, the formulation preferably contains a ratio of alcohol: water of approximately 10:1 to approximately 3:1.

One of ordinary skill in the art will recognize that multiple liquid electric vaporizers exist and may be used with the disclosed air decontamination formulations without departing from the teachings herein. For example, the chimney 206 of the liquid vaporizer 201 may include a chimney extension extending further on the distal side than on the proximal side in order to help direct flow of the vaporized material away from the wall and into the room. The liquid vaporizer 201 may also or alternatively include electronic control means to increase the power that is applied to the heater 202 and, consequently, the rate of emanation of the vaporized air decontamination formulation as disclosed in PCT Pub. No. WO2011/045615 to Reckitt & Colman (Overseas) Limited, the contents of which are incorporated herein in its entirety by reference. The electronic control means may further be programmed to provide sufficient heat for approximately 30 to approximately 60 seconds of vaporization at intermittent time intervals, such as every 4, 8, 12, or 24 hours.

The following example below illustrates exemplary embodiments of the invention. It is to be understood that these examples are provided by way of illustration only and that further embodiments may be produced in accordance with the teachings of the present invention.

### Examples

The following examples were performed in the aerobiological testing chamber 1 as

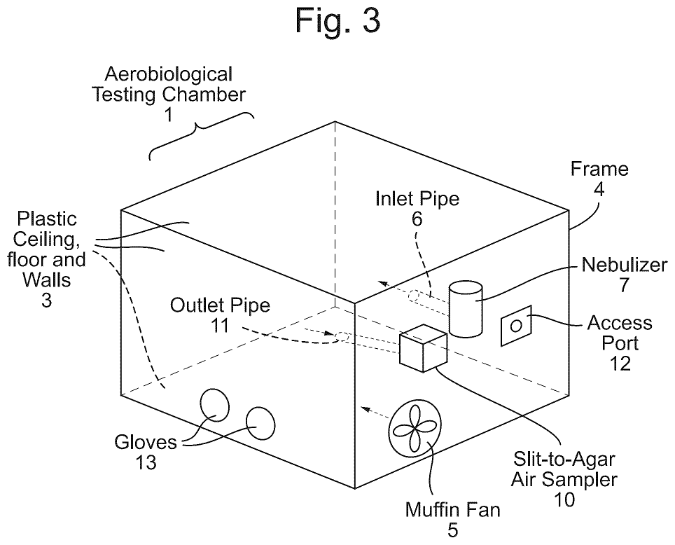

shown in FIG. 3 (which is not to scale). The aerobiological testing chamber 1 has a volume of approximately 24 m³ to approximately 25 m³. The chamber 1 is located inside a clean room (not shown) with negative pressure and control access. The ceiling, floor, and walls of the chamber 1 are made of a wipeable white corrugated plastic sheet 3 affixed to the frame 4 of the chamber 1 to maintain an airtight seal preventing air exchange between the chamber 1 and the clean room. While other colors may be used without departing from the teachings herein, the white corrugated plastic sheets provide better visibility for any residual product stains. The wipeable corrugated plastic sheet 3 used in the examples that follow was 0.0157 inches (0.4 cm) thick and sold at Home Depot under the Coroplast™ brand by Coroplast Fritz Müller Gmbh & Co. The walls should be grounded to dissipate any static electricity that may accumulate. A static electricity discharge wire (not shown) is provided to help discharge any static electricity buildup. The chamber 1 may further include clear plastic windows, sealable access doors, and inlets and outlets for the HEPA filtration system. More detailed depictions of some exemplary chambers 1 may be found in Satter et al., Spread of viral infections by aerosols, Crit Rev Environ Control, 1987, 17:89-131; Sattar et al., Decontamination of indoor air to reduce the risk of airborne infections: Studies on survival and inactivation of airborne pathogens using an aerobiology chamber, American Journal of Infection Control, 44 (2016) e177-e182, the contents of both being incorporated herein by reference in their entireties.

A muffin fan 5, also known as an axial flow fan, is placed on the floor inside the chamber 1 directly underneath the 3.8 cm diameter inlet pipe 6 to a 6-jet nebulizer 7. The 6-jet collision nebulizer 7 generates microbial aerosols in the respirable range of 0.5-5.0 μm. The nebulizer 7 used in the following examples was Model MRE CN24/25 purchased from CH Technologies of Westwood, NJ, US. The nebulizer 7 was connected to a cylinder of extra-dry compressed air with pressure regulator and backflow preventer (neither shown). The fan 5 used was a Nidec Alpha V, TA300, Model A31022-20, Part number 933314 3.0 inch/7.62 m diameter, output 30 CFM supplied by Nidec Corp of Braintree, MA, US. A data recorder (not shown) records the chamber's relative humidity and air temperature. The following examples used the RTR-503L model of wireless data loggers from CAS Data Loggers of Chesterland, OH, US. A magnehelic (not shown) records the pressure differential between the inside and the outside of the chamber 1. The following examples used a magnehelic purchased from ITM Instrument Inc. of Ontario, Canada.

The air in the chamber 1 is sampled at the rate of 1 ft³ (28.3 L)/minute using an externally placed slit-to-agar air sampler with a built-in vacuum pump 10. The sampler 10 used in the examples was purchased from PinPoint Scientific of Bridgend, Wales. The air exiting the sampler 10 is captured in a HEPA filter incorporated in the device 10 or discharged directly into the facility's HEPA-filtered exhaust system (not shown). The sampler 10 draws air samples from the center of the chamber 1 through a 5.0 cm diameter outlet pipe 11.

As discussed by Zargar et al. at page S136, the fan 5 provides uniform distribution of the aerosolized particles in the air inside the chamber 1 when placed at a 45° angle at the bottom of one side of the chamber 1 and operated at 2800 RPM. Mathematical modeling and simulation of bacterial distribution in an aerobiology chamber using computational fluid dynamics, American Journal of Infection Control 44 (2016) S127-S137, incorporated herein in its entirety herein by reference. Zargar et al. further disclose that a 5-minute post-nebulization time is sufficient to distribute introduced bacteria aerosols uniformly throughout the chamber. Id. Zargar et al. further disclose that collection of air samples from the center of the chamber 1 was sufficient to provide a representative profile of the concentration of the airborne bacteria present within the chamber 1. Id.

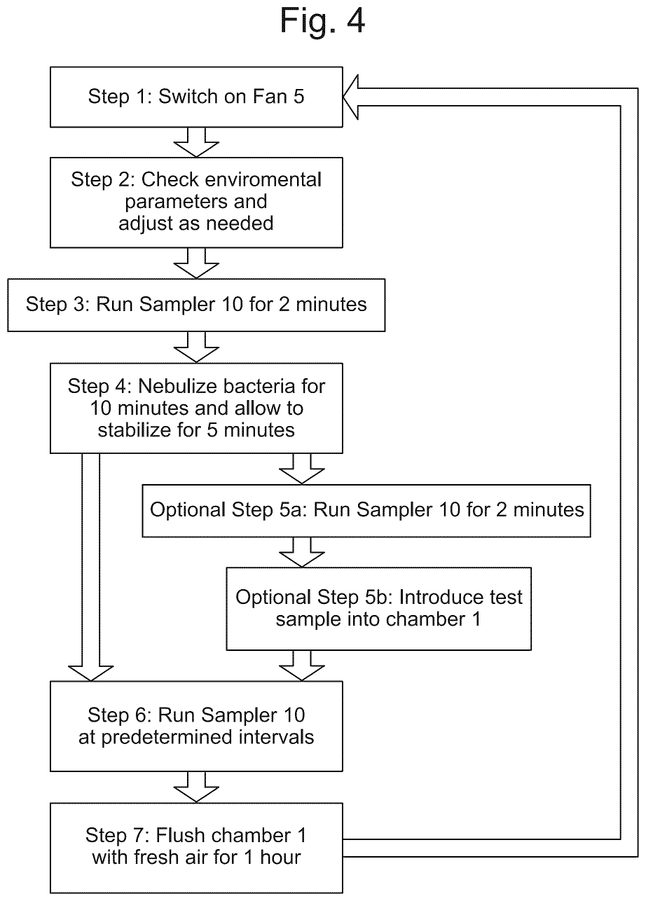

FIG. 4 is a flow chart of the steps performed in the following examples. In step 1, the fan 5 is turned on to start air circulation within the chamber 1. In step 2, the environmental parameters inside the chamber 1 are reviewed and adjusted as needed. Step 3 provides for activation of the slit-to-agar sampler 10 for 2 minutes in order to obtain a sample of any background contamination in the chamber 1. After the sampler 10 is turned off, the bacteria, virus, or phage to be tested is introduced into the chamber 1 via the nebulizer 7 and inlet pipe 6. In this step 4, the nebulizer 7 is run for 10 minutes. The nebulizer 7 is then turned off and the bacteria, virus, or phage allowed to stabilize inside the chamber 1 for 5 minutes. Step 4 should produce a concentration of bacteria, virus, or phage between a minimum of 4.2 log₁₀/m³ to a maximum of 5.0 log₁₀ CFU/m³ in chamber 1.

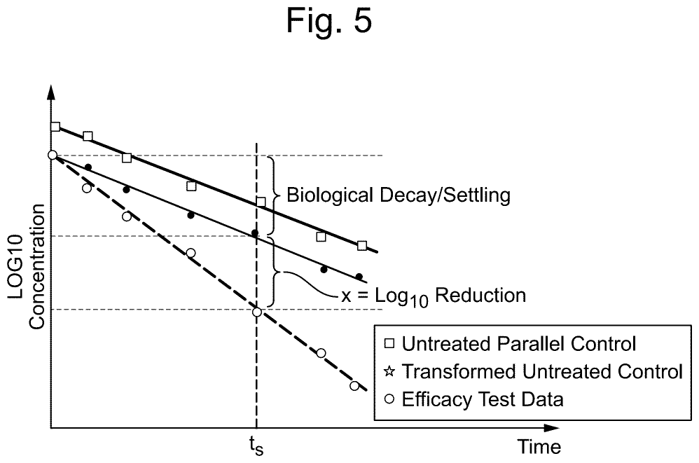

Any meaningful assessment of air decontamination requires that the aerosolized challenge microbes remain viable in the air long enough to allow for proper differentiation between its biological decay or physical fallout and inactivation or removal by the technology being assessed. The test microbes (i.e., bacteria and bacteriophages) were aerosolized into the chamber 1 in Step 4. Steps 5a and 5b are initially skipped to provide the comparative baseline reading of the biological decay or physical fallout. In step 6, samples are collected using a slit-to-agar sampler 10 at predetermined intervals over a specified time frame, for example every 2 minutes over an 8-hour period. The culture plates were incubated at 36° C.±1° C., the colony forming units (CFU) or plaque forming units (PFU) recorded, and the data analyzed to determine the rate of biologic decay. The results are shown in FIG. 5. The chamber 1 was then flushed with fresh air for one hour in Step 7 to decontaminate it and the process started over at Step 1.

Steps 1 to 4 are repeated for different product samples. In Step 5a, the slit-to-agar sampler 10 is run for 2 minutes to determine the initial concentration of the challenge microbes. In Step 5b, the decontamination product to be tested may be introduced into the chamber 1 through an access port 12 in the wall 2 of the chamber 1. Alternatively, the decontamination sample may be placed in the chamber 1 prior to step 1 and accessed and activated using gloves 13 affixed to the wall 2 of the chamber 1 in step 5b. In step 6, samples are collected using a slit-to-agar sampler 10 at predetermined intervals over a specified time frame, for example every 2 minutes over an 8-hour period. The interval and time period for the determination of the biological decay and physical fallout should match the interval and time period used to determine inactivation using the test product.

The culture plates were incubated at 36° C. 35 1° C., the colony forming units (CFU) or plaque forming units (PFU) recorded, and the data analyzed to determine the rate of inactivation. The results are shown in FIG. 5. The chamber 1 was then flushed with fresh air for one hour in Step 7 to decontaminate it and the process started over with a new sample at Step 1.

FIG. 5 is a graph of the log₁₀ concentration of the tested microorganism versus time. The black line shows the rate of biological decay of the challenge bacterial species. The red line shows the rate of inactivation of the challenge bacterial species by the test product. Since the initial titers of the two experiments may differ, the data from the biological decay is transformed so that its initial titer becomes equal to the initial titer for the inactivation test. This transformation step is easily accomplished by one of ordinary skill in the art. The modified biological decay is shown in blue. Successful disinfection results are obtained when the log₁₀ reduction from natural or biological decay to inactivation is equal or greater than 3 log₁₀ after a 5 minute or less contact time. The lyophilized test microbes are obtained from a reputable source, such as the

American Type Culture Collection (ATCC). The microbes are isolated using standard techniques. The bacteria are cultured to provide approximately 1.6×10⁴ CFU/m³ to approximately 1.0×10⁵ CFU/m³. The nebulization fluid is prepared by adding 50 μL of the cultured bacteria, 0.75 mL Bovine Serum Albumin (BSA), 1.05 mL yeast extract, 3.0 mL mucin, and 10 μL of Antifoam A (from Sigma-Aldrich, Cat A-5633) to 10.14 mL of Dulbecco's Phosphate-Buffered Saline (PBS).

The formulations in the following examples were prepared using the ingredients identified in Table A:

TABLE A

| Abbreviation | CAS | Description |
|---|---|---|
| H₂O | 7732- | Deionized Water |
| | 18-5 | |
| CI | 26038- | Corrosion inhibitor, e.g., MEA Borate and |
| | 87-9 | MIPA Borate sold under the tradename |
| | and | Crodacor ™ BE by Croda |
| | 26038- | |
| | 90-4 | |
| MEA | | Monoethanolamine |
| EtOH | 67- | Denatured Ethanol, e.g., SDA 40B sold by |
| | 17-5 | MGP |
| DPG | | DiPropylene Glycol, e.g., Regular Grade |
| | | Dipropylene Glycol sold by Dow |
| TEG | 112- | TriEthylene Glycol |
| | 27-6 | |
| OFX | | PEG-12 Dimethicone, e.g., Xiameter OFX- |
| | | 0193 Fluid sold by Dow |
| SME | | Soyethyl Morpholinium Ethosulfate, e.g., |
| | | Forestall-LQ-(HM) sold by Croda at 35% purity |
| | | in water, alcohol and/or polypropylene glycol |
| Quat | 68989- | Alkyl dimethyl benzyl ammonium saccharinate, |
| | 01-5 | e.g., Oxynide 3300 sold by Stepan |
| F | Various | Fragrance |
| Prop NP-46 | | Propellant blend of 25.86% w/w propane and |
| | | 74.14% w/w N-butane that has a vapor |
| | | pressure of 46 psig |
| Prop | | Propellant blend of 21.02% w/w propane |
| 152a/AB-46 | | and 59.32% w/w isobutane that has a vapor |
| | | pressure of 66 psig |
| Prop A-70 | | Propellant blend of 42.89% w/w propane and |
| | | 57.11% w/w isobutane that has a vapor |
| | | pressure of 70 psig |

The formulations in the following examples were tested using the aerosol nozzles identified in Table B, all of which utilized 2-piece Mechanical Break-up actuators (“2-piece MB”) with swirl chambers.

TABLE B

| | V1 | V2 | V3 | V4 |
|---|---|---|---|---|
| Valve Vapor Tap in | 0.018 | 0.016 | 0.032 | 0.016 |
| inches | | | | |
| [mm] | [0.46] | [0.41] | [0.82] | [0.41] |
| Valve Housing Orifice in | 0.025 | 0.025 | 0.032 | 0.025 |
| inches [mm] | [0.64] | [0.64] | [0.81] | [0.64] |
| Valve Stem Orifice, | 4 × 0.024 | 2 × 0.025 | 4 × 0.024 | 2 × 0.025 |
| quantity × diameter of | [0.61] | [0.64] | [0.61] | [0.64] |
| each orifice in inches | | | | |
| [mm] | | | | |
| Actuator Exit Orifice in | 0.025 | 0.020 | 0.025 | 0.020 |
| inches [mm] | [0.64] | [0.51] | [0.64] | [0.51] |
| Actuator Lan Length in | 0.031 | 0.010 | 0.025 | 0.040 |
| inches [mm] | [0.79] | [0.25] | [0.64] | [1.02] |

### Example 1: S. aureus

R&D samples of the following formulations were tested against Staphylococcus aureus (ATCC 6538).

The formulations and aerosol nozzles used in the testing are provided in Table 1:

TABLE 1

| | | | EtOH | | | | | | | |
|---|---|---|---|---|---|---|---|---|---|---|
| | | | (190 | | | | SME | | | |
| Ref | H₂O | DPG | Proof) | CI | MEA | OFX | (35%) | F | Prop | Valve |
| A | 39.45 | 25 | 10 | 0.1 | 0.1 | 0.05 | 0.2 | 0.1 | 25 152a/ | V3 |
| | | | | | | | (0.07) | | AB-46 | |
| B | 26.6 | 7.5 | 40 | 0.2 | 0.2 | 0.075 | 0.3 | 0.125 | 25 | V1 |
| | | | | | | | (0.1) | | NP-46 | |
| C | 24.1 | 10 | 40 | 0.2 | 0.2 | 0.075 | 0.3 | 0.125 | 25 | V1 |
| | | | | | | | (0.1) | | NP-46 | |
| D | 19.1 | 15 | 40 | 0.2 | 0.2 | 0.075 | 0.3 | 0.125 | 25 | V1 |
| | | | | | | | (0.1) | | NP-46 | |
| E | 19.45 | 25 | 30 | 0.1 | 0.1 | 0.05 | 0.2 | 0.1 | 25 | V4 |
| | | | | | | | (0.07) | | NP-46 | |
| F | 19.45 | 25 | 30 | 0.1 | 0.1 | 0.05 | 0.2 | 0.1 | 25 152a/ | V1 |
| | | | | | | | (0.07) | | AB-46 | |
| G | 19.45 | 25 | 30 | 0.1 | 0.1 | 0.05 | 0.2 | 0.1 | 25 152a/ | V3 |
| | | | | | | | (0.07) | | AB-46 | |
| H | 19.45 | 25 | 30 | 0.1 | 0.1 | 0.05 | 0.2 | 0.1 | 25 152a/ | V4 |
| | | | | | | | (0.07) | | AB-46 | |
| I | 19.45 | 25 | 30 | 0.1 | 0.1 | 0.05 | 0.2 | 0.1 | 25 | V4 |
| | | | | | | | (0.07) | | A-70 | |
| J | 9.45 | 35 | 30 | 0.1 | 0.1 | 0.05 | 0.2 | 0.1 | 25 | V3 |
| | | | | | | | (0.07) | | A-70 | |
| K | 9.45 | 35 | 30 | 0.1 | 0.1 | 0.05 | 0.2 | 0.1 | 25 | V4 |
| | | | | | | | (0.07) | | A-70 | |
| L | 19.53 | 25 | 30 | 0.07 | 0.1 | 0 | 0.2 | 0.1 | 25 | V2 |
| | | | | | | | (0.07) | | A-70 | |
| M | 19.53 | 25 | 30 | 0.07 | 0.1 | 0 | 0.2 | 0.1 | 25 | V4 |
| | | | | | | | (0.07) | | A-70 | |

Spray efficacy was demonstrated by obtaining a 3 log₁₀ reduction of Staphylococcus aureus. When available, the spray time, temperature, percent relative humidity (% RH), and results are provided in Table 2:

TABLE 2

| | | | | | | (a) Log₁₀ | (b) Time | | Spray |
|---|---|---|---|---|---|---|---|---|---|
| | Spray | Wt. | Wt. DPG | | | reduction/ | (minutes)/ | | Visible |
| | Time (In | Formulation | Sprayed | Temp | % | time | 3-LR | Achieve | on |
| Ref | Seconds) | Sprayed (g) | (g) | (° C.) | RH | (minutes) | achieved? | Sanitization? | Floor? |
| A | N/A | N/A | N/A | N/A | N/A | N/A | N/A/No | No | N/A |
| B | 60 | 86.78 | 6.5 | 22.5 | 49 | 4.30/120 | 55/Yes | No | No |
| C | N/A | N/A | N/A | N/A | N/A | N/A | N/A/No | No | N/A |
| D | 10 | 11.24 | 1.7 | 23 | 51 | 0.12/105 | 120/No | No | Yes |
| D | 60 | 51.19 | 7.7 | 22.7 | 54 | 0.40/120 | 120/No | No | Yes |
| E | N/A | N/A | N/A | N/A | N/A | N/A | N/A/No | No | N/A |
| F | 15 | 13.53 | 3.4 | 23 | 51 | 3.31/120 | 65/Yes | No | No |
| F | 15 | 11.53 | 2.9 | 23 | 51 | 0.8/120 | 120/No | No | No |
| F | 15 | 12.78 | 3.2 | 22 | 50 | 1.1/120 | 120/No | No | No |
| F | 15 | 9.65 | 2.4 | 23 | 52 | 1.4/120 | 120/No | No | No |
| F | 30 | 17.29 | 4.3 | 22.5 | 47 | 4.21/120 | 3.70/Yes | Yes | No |
| F | 60 | 60.56 | 15.1 | 23 | 45 | 4.34/120 | 4.37/Yes | Yes | No |
| G | N/A | N/A | N/A | N/A | N/A | N/A | N/A/Yes | Yes | N/A |
| H | N/A | N/A | N/A | N/A | N/A | N/A | N/A/Yes | Yes | N/A |
| I | 15 | 12.81 | 3.2 | 22 | 55 | 1.66/120 | 120/No | No | No |
| I | 20 | 18.77 | 4.7 | 23 | 49 | 2.48/120 | 10/Yes | No | No |
| I | 25 | 20.70 | 5.2 | 21 | 53 | 3.97/120 | 5.17/Yes | No | No |
| I | 25 | 19.92 | 5.0 | 25.4 | 53 | 2.86/120 | 5.75/Yes | No | No |
| I | 30 | 32.77 | 8.2 | 22.5 | 53 | 4.11/120 | 3.91/Yes | Yes | No |
| I | 30 | 22.28 | 5.6 | 24.4 | 51.5 | 3.86/120 | 6.32/Yes | No | No |
| I | 30 | 36.68 | 9.2 | 24.4 | 51.5 | 4.06/120 | 3.91/Yes | Yes | No |
| J | 20 | 18.29 | 6.4 | 25.7 | 54 | 1.70/120 | 120/No | No | No |
| K | N/A | N/A | N/A | N/A | N/A | N/A | N/A/No | No | N/A |
| L | 20 | 26.19 | 6.5 | 24.5 | 55 | 1.5/5 | N/A/No | No* | |
| | | | | | | 2.5/105 | | | |
| L | 30 | 37.67 | 9.4 | 24.5 | 54 | 4.50/120 | 4.12/Yes | No* | No |
| M | N/A | N/A | N/A | N/A | N/A | N/A | N/A/Yes | Yes | N/A |

N/A—this data is not currently available
*Small droplets below the can, but not on floor - may be condensation

Table 2 provides both (a) the time it takes to obtain the specified log₁₀ reduction of Staphylococcus aureus and (b) the time it takes to obtain a 3 logo reduction in Staphylococcus aureus, if indeed a 3 log₁₀ reduction in Staphylococcus aureus was obtained. The formulation is considered a successful air disinfectant when it obtains a 3 log₁₀ reduction in Staphylococcus aureus in 5 minutes or less.

One of ordinary skill in the art will recognize that microbiological test results are not as consistent as chemical test results. Microbiological test results can vary from test to test, even when all other parameters remain the same. Higher variation in microbiological test results is also to be expected in R&D test environments. One of ordinary skill in the art will further recognize that the concentration of bacteria in the air over time may fluctuate. Additionally, the air being sampled in real time may contain residual bacteria when the concentration of the product is sub-optimal/not efficient enough to quickly kill all the bacteria. In other words, multiple factors may have contributed to the results obtained by Formulation I, which was able to achieve a 3 log₁₀ reduction in S. aureus in 5.75 minutes after a 25 second spray, but the reduction decreased to 2.86 log₁₀ after 120 minutes.

As can be seen, the combination of the low concentration of DPG and the low pressure of Formulations B, C, and D did not produce sufficient vapor pressure to achieve sanitization, notwithstanding the higher concentration of ethanol. Formulation E demonstrates that an increase in the concentration of DPG is not sufficient to overcome the limitations of the low-pressure NP-46 propellant. Optimal results were obtained from aerosol formulations containing a ratio of dipropylene glycol:water:alcohol of approximately 1.25:1:1.5.

The approximate 2:1 alcohol:water formulation D produced visible spray on the floor. Applicants believe the propellant may cause the higher concentration of ethanol to evaporate more quickly. No spray was visible on the floor for the aerosol formulations containing a ratio of alcohol:water of approximately 1.5:1.

The 30 second spray of formula F and I and the 60 second spray of formula F successfully achieved air disinfectant germ kill. The 15 second spray results for formula F demonstrate marginal effectiveness that may occasionally generate 3 log₁₀ reduction. In contrast, repeated testing of formula I at 30 seconds produces more consistent effectiveness results.

Table 2 further demonstrates that increasing the spray time (e.g., 60 seconds for Formula B and D) or the percentage of DPG in the formulation (e.g., 35% in Formulations J and K) were not alone sufficient to provide suitable microbiocidal outcome. Formulations J and K were too viscous to move DPG into the vapor phase, even with 70 psig pressure.

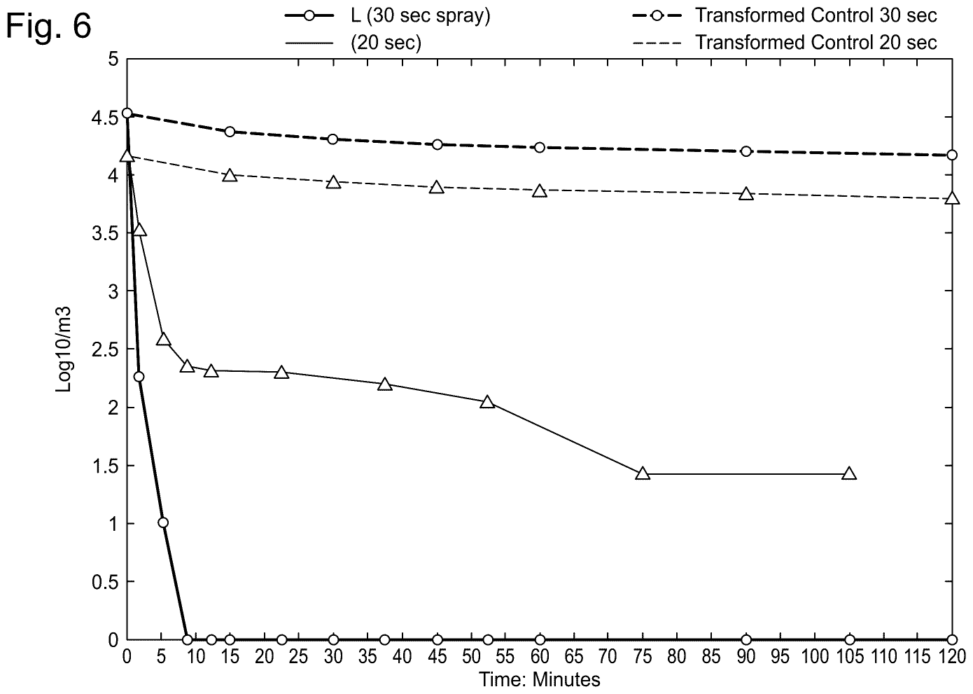

FIG. 6 is a graph of the log₁₀ concentration of S. aureus versus time for Formulation L. As can be seen, both the 20 and 30 second spray times provide fast reduction in S. aureus concentrations.

One of ordinary skill in the art will recognize that a 20- to 30-second spray time is longer than an average consumer expects to depress a spray button. Applicants expect similar efficacy results after multiple but consecutive shorter spray times. For example, ten (10) consecutive 2-or 3-second sprays or four (4) to six (6) consecutive 5-second sprays, with a <1- to 2-second pause between sprays, are expected to produce similar results.

### Example 2: Phi6 and MS2

Spray efficacy was demonstrated by obtaining a 3 log₁₀ reduction of Phi6 or MS2 after a 30 second spray time. The spray time, temperature, percent relative humidity (% RH), and results are provided in Table 3:

TABLE 3

| | | Wt. | | | | | |
|---|---|---|---|---|---|---|---|
| | | Formu- | Wt. | | | Time | Spray |
| | | lation | DPG | | | (minutes) | Visible |
| | | Sprayed | Sprayed | Temp | % | to achieve | on |
| Ref | Phage | (g) | (g) | (° C.) | RH | 3-LR | Floor? |
| J | Phi6 | 29.69 | 7.4 | 24 | 52 | 4.07 | No |
| J | Phi6 | 32.81 | 8.2 | 23.5 | 49.5 | 3.85 | No |
| J | MS2 | 33.28 | 8.3 | 23.4 | 54 | 14.09 | No |
| J | MS2 | 26.77 | 6.7 | 22.8 | 49 | 13.57 | No |

As discussed above, Table 3 confirms that non-enveloped viruses like MS2 are more difficult to inactivate than bacteria and enveloped viruses like Phi6. That notwithstanding, a 30-second spray of the Formulation J is still capable of providing a 3 log₁₀ reduction in concentration.

### Example 3: DPG vs TEG

Four (4) formulations in Table 4 were tested against S. epidermidis to evaluate the efficacy of DPG versus TEG: TEG Only, DPG Only, TEG-DPG Blend, and TEG-Based. S. epidermidis is a safer, yet equally as relevant as S. aureus, surrogate for a variety of vegetative nosocomial pathogens with potential for airborne spread. Approximately 200 g of product was introduced into the chamber 1 (i.e., the entire canister).

TABLE 4

| | TEG Only | DPG Only | TEG-DPG Blend | TEG-Based |
|---|---|---|---|---|
| H2O | 36.8 | 36.8 | 33.8 | 49.03 |
| DPG | 0 | 3 | 3 | 0 |
| TEG | 3 | 0 | 3 | 5.4 |
| CI | 0.2 | 0.2 | 0.2 | 0.1 |
| EtOH | 40 | 40 | 40 | 20.42 |
| Quat | 0 | 0 | 0 | 0.15 |
| Prop | 20 | 20 | 20 | 24.9 |

The DPG Only and TEG-DPG formulations showed a mean ≥3 log₁₀ reduction from a mean baseline titer of 4.56 log₁₀ after a ≤10 minute exposure. The TEG Only formulation showed a mean log₁₀ reduction of 1.33 at 10 minutes and 2.35 at 60 minutes from a mean baseline titer of 4.83 log 10. The TEG-Based formulation showed a mean log₁₀ reduction of 1.59 at 10 minutes and 2.93 at 60 minutes from a mean baseline titer for 4.36 log10. This data demonstrates that DPG is a more effective microbicide in the air than TEG.

The foregoing description of various aspects of the invention has been presented for purposes of illustration and description. Embodiments and/or features therein may be freely combined with one another. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

1. An air decontamination formulation comprising:
approximately 28% to approximately 38% by weight of the formulation of dipropylene glycol;
approximately 0.12% to approximately 0.43% by weight of the formulation of an odor neutralizing cationic surfactant;
approximately 28% to approximately 50% by weight of the formulation of an alcohol; and
approximately 14% to approximately 33% by weight of the formulation of water.

2. (canceled)

3. (canceled)

4. The air decontamination formulation of claim 1, wherein the formulation is a single phase liquid.

5. (canceled)

6. (canceled)

7. (canceled)

8. (canceled)

9. (canceled)

10. The air decontamination formulation of claim 1, wherein the formulation does not include an emulsifier, a nonionic surfactant, an amphoteric surfactant, or any combination thereof.

11. The air decontamination formulation of claim 1, the formulation further comprising a corrosion inhibitor.

12. A method of sanitizing air in 0.1 to 5 minutes, the method comprising introducing between approximately 0.825 g/second to approximately 1.125 g/second of the air decontamination formulation of claim 1 into the air for approximately 30 to approximately 60 seconds, the air occupying a volume being less than or equal to 25 m³.

13. An air decontamination product comprising
an aerosol canister containing a 2-piece mechanical breakup (MB) nozzle with swirl chamber, approximately 70% to approximately 80% by weight of the combined air decontamination formulation and aerosol of the air decontamination formulation of claim 1, and approximately 20% to approximately 30% by weight of combined air decontamination formulation and aerosol of a pressurized liquified petroleum gas,
the pressurized liquified petroleum gas comprising a blend of propane and butane sufficient to produce at least 60 psig pressure;
the MB nozzle comprising a housing orifice having a diameter ranging from approximately 0.02 inches [0.5 mm] to approximately 0.03 inches [0.8 mm], a stem vapor tap having a diameter ranging from approximately 0.016 inches [0.41 mm] to approximately 0.020 inches [0.51 mm], and a quantity of two [2] or four [4] stem orifices having a diameter ranging from approximately 0.024 inches [0.61 mm] to approximately 0.025 inches [0.64 mm].

14. The air decontamination product of claim 13, wherein the pressurized liquified petroleum gas comprises approximately 40% to approximately 60% by weight of the gas of propane and approximately 60% to approximately 40% by weight of the gas of isobutane.

15. The method of claim 12, wherein the air decontamination formulation of claim 1 is contained in the air decontamination product of claim 13,
the method comprising
actuating the MB nozzle of the air decontamination product of claim 13 to introduce between approximately 1.1 g/second to approximately 1.5 g/second of the air decontamination formulation into the air for approximately 30 to approximately 60 seconds, the air occupying a volume being less than or equal to 25 m³.

16. The air decontamination formulation of claim 1, wherein the odor neutralizing cationic surfactant is a quaternary morpholinium alkyl sulfate compound selected from: soyethyl morpholinium ethosulfate, cetethyl morpholinium ethosulfate, N-myristyl-N-methyl morpholinium methyl sulfate, and N-oleyl-N-methyl morpholinium methyl sulfate.

17. The method of claim 12, wherein the air decontamination formulation of claim 1 is a single phase liquid.

18. The method of claim 12, wherein the air decontamination formulation of claim 1 wherein the odor neutralizing cationic surfactant is a quaternary morpholinium alkyl sulfate compound selected from: soyethyl morpholinium ethosulfate, cetethyl morpholinium ethosulfate, N-myristyl-N-methyl morpholinium methyl sulfate, and N-oleyl-N-methyl morpholinium methyl sulfate.

19. The method of claim 12, wherein the air decontamination formulation of claim 1 does not include an emulsifier, a nonionic surfactant, an amphoteric surfactant, or any combination thereof.

20. The method of claim 12, wherein the air decontamination formulation of claim 1 further comprises a corrosion inhibitor.

21. The air decontamination product of claim 13, wherein the air decontamination formulation of claim 1 is a single phase liquid.

22. The air decontamination product of claim 13, wherein the air decontamination formulation of claim 1 wherein the odor neutralizing cationic surfactant is a quaternary morpholinium alkyl sulfate compound selected from: soyethyl morpholinium ethosulfate, cetethyl morpholinium ethosulfate, N-myristyl-N-methyl morpholinium methyl sulfate, and N-oleyl-N-methyl morpholinium methyl sulfate.

23. The air decontamination product of claim 13, wherein the air decontamination formulation of claim 1 does not include an emulsifier, a nonionic surfactant, an amphoteric surfactant, or any combination thereof.

24. The air decontamination product of claim 13, wherein the air decontamination formulation of claim 1 further comprises a corrosion inhibitor.

25. The method of claim 15, wherein the air decontamination formulation of claim 1 is a single phase liquid.

26. The method of claim 15, wherein the air decontamination formulation of claim 1 wherein the odor neutralizing cationic surfactant is a quaternary morpholinium alkyl sulfate compound selected from: soyethyl morpholinium ethosulfate, cetethyl morpholinium ethosulfate, N-myristyl-N-methyl morpholinium methyl sulfate, and N-oleyl-N-methyl morpholinium methyl sulfate.

27. The method of claim 15, wherein the air decontamination formulation of claim 1 does not include an emulsifier, a nonionic surfactant, an amphoteric surfactant, or any combination thereof.
